(19) United States
(12) Patent Application Publication
(10) Pub. No.: US 2004/0019259 A1
(43) Pub. Date: Jan. 29, 2004

(54) Remote monitoring and data management platform

(76) Inventors: Stephen J. Brown, Woodside, CA (US); Julie C. Cherry, Milpitas, CA (US); Geoffrey Clapp, Los Altos, CA (US)

Correspondence Address: BLACK LOWE & GRAHAM, PLLC, 701 FIFTH AVENUE, SUITE 4800, SEATTLE, WA, 98104, US

(21) Appl. No.: 10/435,619

(22) Filed: May 8, 2003

Related U.S. Application Data
(63) Continuation-in-part of application No. 10/233,296, filed on Aug. 30, 2002.
Application No. 10/233,296 is a continuation-in-part of application No. 09/665,442, filed on Sep. 19, 2000.
Application No. 09/665,442 is a continuation-in-part of application No. 09/357,536, filed on Jul. 19, 1999, now abandoned.
Application No. 09/357,536 is a continuation of application No. 09/127,404, filed on Jul. 31, 1998, now Pat. No. 5,940,801.
Application No. 09/127,404 is a continuation of application No. 08/843,495, filed on Apr. 16, 1997, now Pat. No. 5,828,943.
Application No. 08/843,495 is a continuation of application No. 08/682,385, filed on Jul. 17, 1996, now abandoned.
Application No. 08/682,385 is a continuation of application No. 08/479,570, filed on Jun. 7, 1995, now abandoned.
Application No. 08/479,570 is a continuation of application No. 08/233,674, filed on Apr. 26, 1994, now abandoned.
(63) Continuation-in-part of application No. 10/279,749, filed on Oct. 23, 2002.
Application No. 10/279,749 is a continuation-in-part of application No. 10/233,296, filed on Aug. 30, 2002.
Application No. 10/233,296 is a continuation-in-part of application No. 09/713,922, filed on Nov. 15, 2000, now abandoned.
Application No. 09/713,922 is a continuation-in-part of application No. 08/975,774, filed on Nov. 21, 1997, now Pat. No. 6,101,478.
Application No. 10/233,296 is a continuation-in-part of application No. 09/665,442, filed on Sep. 19, 2000.
Application No. 09/665,442 is a continuation-in-part of application No. 09/293,363, filed on Apr. 16, 1999, now abandoned.
Application No. 09/665,442 is a continuation-in-part of application No. 09/203,882, filed on Dec. 1, 1998, now abandoned.
Application No. 09/665,442 is a continuation-in-part of application No. 09/159,219, filed on Sep. 23, 1998, now abandoned.
Application No. 09/665,442 is a continuation-in-part of application No. 09/159,058, filed on Sep. 23, 1998, now abandoned.
Application No. 09/665,442 is a continuation-in-part of application No. 09/203,880, filed on Dec. 1, 1998, now abandoned.
Application No. 09/665,442 is a continuation-in-part of application No. 09/201,323, filed on Nov. 30, 1998, now abandoned.
Application No. 09/665,442 is a continuation-in-part of application No. 09/320,004, filed on May 26, 1999, now abandoned.
Application No. 09/665,442 is a continuation-in-part of application No. 09/318,708, filed on May 26, 1999, now abandoned.
Application No. 09/665,442 is a continuation-in-part of application No. 09/274,433, filed on Jun. 17, 1999, now abandoned.
Application No. 09/665,442 is a continuation-in-part of application No. 09/422,046, filed on Oct. 20, 1999.
Application No. 09/422,046 is a continuation of application No. 09/271,217, filed on Mar. 17, 1999, now Pat. No. 6,168,563.
Application No. 09/271,217 is a continuation-in-part of application No. 08/481,925, filed on Jun. 7, 1995, now Pat. No. 5,899,855.
Application No. 08/481,925 is a continuation of application No. 08/233,397, filed on Apr. 26, 1994, now abandoned.
Application No. 08/481,925 is a continuation-in-part of application No. 07/977,323, filed on Nov. 17, 1992, now Pat. No. 5,307,263.
Application No. 09/422,046 is a continuation-in-part of application No. 08/946,341, filed on Oct. 7, 1997, now Pat. No. 5,997,476.
Application No. 08/946,341 is a continuation-in-part of application No. 08/847,009, filed on Apr. 30, 1997, now Pat. No. 5,897,493.
Application No. 10/233,296 is a continuation-in-part of application No. 09/237,194, filed on Jan. 26, 1999.
Application No. 09/237,194 is a continuation of application No. 08/481,925, filed on Jun. 7, 1995, now Pat. No. 5,899,855.
Application No. 08/481,925 is a continuation of application No. 08/233,397, filed on Apr. 26, 1994, now abandoned.
Application No. 08/233,397 is a continuation-in-part of application No. 07/977,323, filed on Nov. 17, 1992, now Pat. No. 5,307,263.
Application No. 10/233,296 is a continuation-in-part of application No. 09/201,372, filed on Nov. 30, 1998, now abandoned.
Application No. 09/201,372 is a continuation-in-part of application No. 08/669,613, filed on Jun. 24, 1996, now Pat. No. 5,879,163.
Application No. 10/233,296 is a continuation-in-part of application No. 09/378,188, filed on Aug. 20, 1999, now abandoned.
Application No. 09/378,188 is a continuation of application No. 08/850,840, filed on May 3, 1997, now Pat. No. 5,985,559.
Application No. 08/850,840 is a continuation-in-part of application No. 08/847,009, filed on Apr. 30, 1997, now Pat. No. 5,897,493.
Application No. 10/233,296 is a continuation-in-part of application No. 09/304,447, filed on May 3, 1999, now abandoned.
Application No. 09/304,447 is a continuation of application No. 08/771,951, filed on Dec. 23, 1996, now Pat. No. 5,933,136.
Application No. 10/233,296 is a continuation-in-part of application No. 09/531,237, filed on Mar. 21, 2000, now abandoned.
Application No. 09/531,237 is a continuation-in-part of application No. 09/300,856, filed on Apr. 28, 1999, now Pat. No. 6,368,273.
Application No. 09/300,856 is a division of application No. 08/946,341, filed on Oct. 7, 1997, now Pat. No. 5,997,476.
Application No. 08/946,341 is a continuation-in-part of application No. 08/847,009, filed on Apr. 30, 1997, now Pat. No. 5,897,493.
Application No. 10/233,296 is a continuation-in-part of application No. 09/540,482, filed on Mar. 31, 2000.
Application No. 09/540,482 is a continuation of application No. 09/394,219, filed on Sep. 13, 1999, now Pat. No. 6,375,469.
Application No. 09/394,219 is a continuation of application No. 08/814,293, filed on Mar. 10, 1997, now Pat. No. 5,951,300.
Application No. 10/233,296 is a continuation-in-part of application No. 09/518,426, filed on Mar. 3, 2000, now abandoned.
Application No. 09/518,426 is a continuation of application No. 08/972,375, filed on Nov. 18, 1997, now Pat. No. 6,068,615.
Application No. 08/972,375 is a continuation-in-part of application No. 08/681,290, filed on Jul. 22, 1996, now Pat. No. 5,782,814.
Application No. 08/681,290 is a continuation-in-part of application No. 08/278,929, filed on Jul. 22, 1994, now Pat. No. 5,569,212.
Application No. 09/665,442 is a continuation-in-part of application No. 09/495,809, filed on Feb. 1, 2000, now abandoned.
Application No. 09/495,809 is a continuation of application No. 09/271,188, filed on Mar. 17, 1999, now Pat. No. 6,334,778.
Application No. 09/271,188 is a continuation-in-part of application No. 09/127,404, filed on Jul. 31, 1998, now Pat. No. 5,940,801.
Application No. 09/127,404 is a continuation of application No. 08/843,495, filed on Apr. 16, 1997, now Pat. No. 5,828,943.
Application No. 08/843,495 is a continuation of application No. 08/682,385, filed on Jul. 17, 1996, now abandoned.
Application No. 08/682,385 is a continuation of application No. 08/479,570, filed on Jun. 7, 1995, now abandoned.
Application No. 08/479,570 is a continuation of application No. 08/233,674, filed on Apr. 26, 1994, now abandoned.
Application No. 10/233,296 is a continuation-in-part of application No. 09/336,570, filed on Jun. 21, 1999, now Pat. No. 6,186,145.
Application No. 09/336,570 is a continuation-in-part of application No. 08/958,786, filed on Oct. 29, 1997, now Pat. No. 5,913,310.
Application No. 08/958,786 is a continuation-in-part of application No. 08/857,187, filed on May 15, 1997, now Pat. No. 5,918,603.
Application No. 08/857,187 is a continuation of application No. 08/247,716, filed on May 23, 1994, now Pat. No. 5,678,571.
Application No. 09/495,809 is a continuation of application No. 09/271,188, filed on Mar. 17, 1999, now Pat. No. 6,334,778.
Application No. 09/271,188 is a continuation-in-part of application No. 09/127,404, filed on Jul. 31, 1998, now Pat. No. 5,940,801.
Application No. 09/127,404 is a continuation of application No. 08/843,495, filed on Apr. 16, 1997, now Pat. No. 5,828,943.
Application No. 08/843,495 is a continuation of application No. 08/682,385, filed on Jul. 17, 1996, now abandoned.
Application No. 08/682,385 is a continuation of application No. 08/479,570, filed on Jun. 7, 1995, now abandoned.
Application No. 08/479,570 is a continuation of application No. 08/233,674, filed on Apr. 26, 1994, now abandoned.
Application No. 09/378,188 is a continuation-in-part of application No. 08/946,341, filed on Oct. 7, 1997, now Pat. No. 5,997,476.
Application No. 08/946,341 is a continuation-in-part of application No. 08/847,009, filed on Apr. 30, 1997, now Pat. No. 5,897,493.
Application No. 09/271,188 is a continuation-in-part of application No. 09/127,404, filed on Jul. 31, 1998, now Pat. No. 5,940,801.
Application No. 09/127,404 is a continuation of application No. 08/843,495, filed on Apr. 16, 1997, now Pat. No. 5,828,943.
Application No. 08/843,495 is a continuation of application No. 08/682,385, filed on Jul. 17, 1996, now abandoned.
Application No. 08/682,385 is a continuation of application No. 08/479,570, filed on Jun. 7, 1995, now abandoned.
Application No. 08/479,570 is a continuation of application No. 08/233,674, filed on Apr. 26, 1994, now abandoned.
Application No. 10/233,296 is a continuation-in-part of application No. 09/496,893, filed on Feb. 2, 2000.
Application No. 09/496,893 is a continuation of application No. 09/041,809, filed on Mar. 13, 1998, now abandoned.
Application No. 10/233,296 is a continuation-in-part of application No. 09/658,209, filed on Sep. 8, 2000.
Application No. 09/658,209 is a continuation-in-part of application No. 09/237,194, filed on Jan. 26, 1999.
Application No. 09/237,194 is a continuation of application No. 08/481,925, filed on Jun. 7, 1995, now Pat. No. 5,899,855.
Application No. 08/481,925 is a continuation of application No. 08/233,397, filed on Apr. 26, 1994, now abandoned.
Application No. 08/233,397 is a continuation-in-part of application No. 07/977,323, filed on Nov. 17, 1992, now Pat. No. 5,307,263.
Application No. 10/233,296 is a continuation-in-part of application No. 09/160,970, filed on Sep. 25, 1998, now Pat. No. 6,240,393.
Application No. 09/160,970 is a continuation-in-part of application No. 09/092,604, filed on Jun. 5, 1998, now Pat. No. 6,023,686.
Application No. 09/092,604 is a continuation-in-part of application No. 08/784,270, filed on Jan. 15, 1997, now Pat. No. 5,887,133.
Application No. 09/092,604 is a continuation-in-part of application No. 08/603,131, filed on Feb. 20, 1996, now Pat. No. 5,794,219.
Application No. 10/233,296 is a continuation-in-part of application No. 09/880,735, filed on Jun. 12, 2001, now abandoned.
Application No. 09/880,735 is a continuation of application No. 09/152,353, filed on Sep. 14, 1998, now Pat. No. 6,246,992.
Application No. 09/152,353 is a continuation-in-part of application No. 08/732,158, filed on Oct. 16, 1996, now Pat. No. 5,832,448.
Application No. 10/233,296 is a continuation-in-part of application No. 09/653,664, filed on Sep. 1, 2000.
Application No. 09/653,664 is a continuation of application No. 09/304,446, filed on May 3, 1999, now Pat. No. 6,167,386.
Application No. 09/304,446 is a continuation of application No. 09/092,604, filed on Jun. 5, 1998, now Pat. No. 6,023,686.
Application No. 09/092,604 is a continuation-in-part of application No. 08/603,131, filed on Feb. 20, 1996, now Pat. No. 5,794,219.
Application No. 09/092,604 is a continuation-in-part of application No. 08/784,270, filed on Jan. 15, 1997, now Pat. No. 5,887,133.
Application No. 10/233,296 is a continuation-in-part of application No. 09/441,408, filed on Nov. 16, 1999, now abandoned.
Application No. 10/233,296 is a continuation-in-part of application No. 09/810,334, filed on Mar. 14, 2001.
Application No. 10/233,296 is a continuation-in-part of application No. 09/810,865, filed on Mar. 16, 2001.
Application No. 09/810,865 is a continuation of application No. 09/399,122, filed on Sep. 20, 1999, now Pat. No. 6,233,539.
Application No. 09/399,122 is a continuation of application No. 08/781,278, filed on Jan. 10, 1997, now Pat. No. 5,956,501.
Application No. 10/233,296 is a continuation-in-part of application No. 10/024,445, filed on Dec. 17, 2001.
Application No. 10/233,296 is a continuation-in-part of application No. 09/799,852, filed on Mar. 5, 2001.
Application No. 09/799,852 is a continuation of application No. 09/274,431, filed on Mar. 22, 1999, now Pat. No. 6,196,970.
Application No. 09/799,852 is a continuation-in-part of application No. 09/119,546, filed on Jul. 20, 1998, now Pat. No. 6,330,426.
Application No. 09/119,546 is a continuation-in-part of application No. 08/953,883, filed on Oct. 20, 1997, now abandoned.
Application No. 08/953,883 is a continuation-in-part of application No. 08/757,129, filed on Dec. 3, 1996, now Pat. No. 6,144,837.
Application No. 08/757,129 is a continuation-in-part of application No. 08/334,643, filed on Nov. 4, 1994, now Pat. No. 5,601,435.
Application No. 10/233,296 is a continuation-in-part of application No. 08/995,609, filed on Dec. 22, 1997, now Pat. No. 6,210,272.
(60) Provisional application No. 60/041,746, filed on Mar. 28, 1997.
(60) Provisional application No. 60/041,746, filed on Mar. 28, 1997.
(60) Provisional application No. 60/041,751, filed on Mar. 28, 1997.
(60) Provisional application No. 60/041,746, filed on Mar. 28, 1997.
(60) Provisional application No. 60/041,751, filed on Mar. 28, 1997.
(60) Provisional application No. 60/041,746, filed on Mar. 28, 1997.
(60) Provisional application No. 60/041,751, filed on Mar. 28, 1997.
(60) Provisional application No. 60/189,536, filed on Mar. 15, 2000.
(60) Provisional application No. 60/256,715, filed on Dec. 18, 2000.
(60) Provisional application No. 60/336,198, filed on Oct. 23, 2001.
(60) Provisional application No. 60/336,198, filed on Oct. 23, 2001.
(60) Provisional application No. 60/326,521, filed on Oct. 1, 2001.

(51) Int. Cl. A61B 5/00

(52) U.S. Cl. 600/300

(57) ABSTRACT

A networked system for remotely determining the health status of an individual or a plurality of individuals and to remotely communicate intervention protocols to an individual or a plurality of individuals using microprocessor controlled communication devices.

### PRIORITY CLAIM

This application claims the benefit of U.S. provisional patent application serial No. 60/379,330, filed May 8, 2002, which is hereby incorporated by reference.

### FIELD OF THE INVENTION

This invention relates generally to remote health monitoring systems, as applied to patients and health care providers to establish an easy deployable, proactive patient management and communication platform to timelier monitor and manage patient disease. In particular, the invention relates to a multi-user remote health monitoring system that is capable of reliably identifying and interactively querying patients, healthcare providers, and healthcare analysts across remote systems to ascertain current health status of patients and to timely communicate health maintenance and treatment related information to the patient.

### BACKGROUND OF THE INVENTION

In the traditional, treatment-based model of care, patients only interact with their care providers at scheduled appointments, or when their health status has deteriorated to the point of necessitating a trip to the emergency room or hospital. The patient's deteriorated health status results in a more expensive treatment and time-consuming recovery. This is particularly true with demographic analyses showing how aging populations exhibit a major drain of health care resources as chronic diseases manifests by more visits to doctors offices, clinics, hospitals, and emergency rooms. Furthermore, all population groups, the young, middle-age, and the elderly, face new health threats including increased drug resistance to mutating infectious agents, and the looming threat of bioterrorism based events. Finally, diminishing government support to Medicare and Medicaid stresses the importance of having a remote monitoring and data management platform to rapidly identify patient disease and offer timely medical interventions to minimize individual patient health deterioration and to limit the deterioration of health in population groups.

Critical gaps exist in our nation's ability to anticipate, detect and respond to epidemics that may be ‘natural’ or that which may be attributed to bioterrorism due to ineffective disease surveillance and fragmented information systems. Federal, state, and local agencies urgently need (i) surveillance capabilities to accelerate time-to-detection and monitor preparedness at the local level, and (ii) integrated information solutions that enable rapid statistical analyses and facilitate investigations.

Surveillance systems should monitor the health of the population in real time and on a continuous basis. Many medical conditions offer only a brief window between diagnosis and treatment. In addition, many infectious diseases, for example, anthrax, cannot be successfully treated once the condition has become advanced but can be treated if caught early. Thus, the window of time during which effective intervention is possible can be very narrow.

In order to facilitate rapid responses, surveillance systems must be capable of monitoring information on an ongoing basis. In order to intervene successfully to acute and chronic medical conditions and to prevent the onset of new ones requires the existence of a health surveillance system capable of providing a continuous, real-time (or as near real-time as possible), and accurate overview of a population's health.

### SUMMARY OF THE INVENTION

The invention presents a networked system for remotely identifying, querying and monitoring the health related parameters of a plurality of individuals who are homebound, at healthcare facilities and their respective healthcare providers, by a two-way multiple cross platform communication system. The system includes a server and a workstation for entering into the server query sets to be answered by the individuals using personal digital assistants (PDA) based computers, laptop computers, desktop computers, and cellular telephones. The server is preferably a World Wide Web server and the workstation is preferably a personal computer or network terminal connected to the web server via a secure Internet connection. The system also includes a remotely programmable apparatus for identifying and interacting with the individuals. The remotely programmable apparatus is connected to the server via a communication network, preferably a secure Internet connection. The remotely programmable apparatus interacts with the individuals in accordance with script programs received from the server.

The server includes a script generator for generating script programs from the query sets, which are entered through a client station. The script programs are developed at the client station and deployed across the multi platform network. The scripts are reconfigurable and responsive to incoming answers executed by the microprocessor-based remotely programmable apparatuses, PDAs, computers, and cellular telephones. The client station communicates the query sets to the individuals, receives responses to the query sets, and to transmits the responses from the remotely programmable apparatus to the server. The server includes a database connected to the script generator for storing the script program, and the responses to the queries. The database also stores a list of individuals or individual types, and for each individual or individual type, has a pointer to at least one script program. The server also has a report generator, an analytical engine that can retrieve stored responses and other information from the database, follow up on specific bits of information that fulfills certain criteria as determined by the operator, organize and numerically manipulate the dataset for the purpose of analysis, and represent the information contained therein in a pictorial form, such as a graph or chart, etc.

The remotely programmable apparatus has a communication device, such as a modem, for receiving the script programs from the server and for transmitting the responses to the server. The remotely programmable apparatus also has a user interface for communicating the query sets to the individuals and for receiving the responses to the query sets. In the preferred embodiment, the user interface includes a display for displaying the query sets and user input buttons for entering the responses to the query sets.

The remotely programmable apparatus also includes a memory for storing the script programs and the responses to the query sets. The remotely programmable apparatus further includes a microprocessor connected to the communication device, the user interface, and the memory. The microprocessor executes the script programs to identify the individual, communicate the query sets to the individual, receive the responses to the query sets, and transmit the responses to the server through the communication network.

In one preferred embodiment, the system also includes at least one monitoring device for producing measurements of a health condition or parameter of a patient and for transmitting the measurements to the apparatus. The monitoring device can also be used to help the remotely programmable apparatus identify the individual operator. The remotely programmable apparatus includes a device interface connected to the microprocessor for receiving the measurements and information from the monitoring device. The measurements are stored in the memory and transmitted to the server along with the operator's identity and the responses to the query sets. The server also preferably includes a report generator connected to the database for generating a report of the measurements and responses. The report is displayed on the workstation.

In another preferred embodiment, the system includes a monitoring application that is capable of acquiring data (responses and measurements) from either the remote apparatus or the database and use this data to select another script to be sent to one or more monitoring devices, not necessarily the one from which it received the said data, according to criteria that have been established in advance by the public health workers and/or healthcare providers.

As the present invention has multi-user capabilities, it is able to identify each individual or individual type in order to select the correct script program. In one preferred embodiment, the individual can enter his or her unique identification code into the remotely programmable apparatus. The code is sent to the server and used to determine which script program to send back to the apparatus.

In another preferred embodiment, the system uses a data card, which contains information about an individual's identity. The remotely programmable apparatus includes a data card reader in which the data card can be placed and read. A personal identification number (PIN) can also be used in conjunction with the data card in order confirm an individual's identity. In this preferred embodiment, the present invention resembles an ATM machine.

The system of the present invention can also identify an individual by intercepting data from a separate information system. Data sent from a server of the separate information system to a printer can pass through the remotely programmable apparatus, which can identify the individual and send the data to the server of the present invention. Data passing through the remotely programmable apparatus can also trigger a script program, which can display queries for the individual to answer, or send information to the printer to be printed. An example of this preferred embodiment has the remotely programmable apparatus located in series between an information system server and an information system printer. Alternatively, this data can also include that entered on the keyboard, that which is stored in the RAM, or that which is stored in a given set of files or a folder information system server. In any case, the data is captured by a software program and analyzed using the same set of rules as that above, to trigger a script on the apparatus.

In addition, the system would also have the capability of collecting data from a range of sources, such as Interactive Voice Response Systems, Internet based forms, wireless devices and the invention enabled monitoring appliances, and integrating the above data into the database, after conversion into the standardized format.,

Furthermore, the server has multiple software programs to establish remote patient education; health condition data stratification; psychological diagnostic assessments and interventions; health behavior rewards and incentives; integration of health and entertainment media; disease modeling and predictive algorithms, including diabetes, cardiovascular, respiratory, weight loss, fitness, stress, smoking cessation, and mental health; content filtering systems and methods,

Finally, the data analysis, methods, criteria for data segregation, and techniques and methods to automate several of the functions of the operator, in order to achieve quicker response times, reduced administration costs and reduction in the possibility of errors are described.

### BRIEF DESCRIPTION OF THE DRAWINGS

FIG. 1 is a block diagram of a networked system according to a preferred embodiment of the invention.

FIG. 2 is a block diagram illustrating the two-way interaction of the components of the system of FIG. 1.

FIG. 3 is a block diagram of a preferred embodiment where a plurality of remotely programmable apparatuses communicate to a client station via an Internet server;

FIG. 4 is a diagram illustrating the remotely programmable device as used for chronic care of a patient in a home setting;

FIG. 5 is a diagram illustrating the remotely programmable device as used for health surveillance in a hospital;

FIG. 6 is an illustration of script entry inputs to a mobile or cellular phone;

FIG. 7 is an illustration of digital screen shots in a preferred embodiment of the invention using digital-based or Web television;

FIG. 8 is an illustration of medical devices in communicating with the remotely programmable device;

FIG. 9 is a home screen shot as would appear on the client station preferred embodiment of the invention;

FIG. 10 is screen shot of patient work list as would appear on the client station preferred embodiment of the invention;

FIG. 11 is a screen shot of a patient results report as would appear on the client station preferred embodiment of the invention;

FIG. 12 is a screen shot of a trend report as would appear on the client station preferred embodiment of the invention;

FIG. 13 is a screen shot of patients' notes as would appear on the client station preferred embodiment of the invention;

FIG. 14 is a screen shot of a trend report of an individual patient as would appear on the client station preferred embodiment of the invention;

FIG. 15 is a screen shot of a survey questionnaire to an individual patient as appearing on the patient's personal computer in a web-preferred embodiment of the invention;

FIG. 16 is a screen shot of a trend chart to an individual patient as appearing on the patient's personal computer in the web-preferred embodiment of the invention;

FIG. 17 is a screen shot of a “view my results” report as appearing on the patient's personal computer in the web-preferred embodiment of the invention;

FIG. 18 is a screen shot of a “Special Report” as appearing on the patient's personal computer in the web-preferred embodiment of the invention;

FIG. 19 is a screen shot of a “Trends-Weight Control” as appearing on the patient's personal computer and the client station in the web-preferred embodiment of the invention;

FIG. 20 is a screen shot of a “Trends-Rhythm Strip” as appearing on the patient's personal computer and the client station in the web-preferred embodiment of the invention; and

FIG. 21 is a screen shot of a “Trends-Blood Pressure and Pulse” as appearing on the patient's personal computer and the client station in the web-preferred embodiment of the invention.

### DETAILED DESCRIPTION OF THE PREFERRED EMBODIMENT

This application incorporates by reference, in their entirety as though set forth fully herein, each of the applications listed below:

U.S. patent application Ser. No. 10/356,720 filed Jan. 30, 2003.

U.S. patent application Ser. No. 10/319,427 filed Dec. 12, 2002.

U.S. patent application Ser. No. 10/279,749 filed Oct. 23, 2002.

U.S. patent application Ser. No. 10/233,296 filed Aug. 30, 2002.

U.S. patent application Ser. No. 09/971,785 filed Oct. 4, 2001.

U.S. patent application Ser. No. 09/810,856 filed Mar. 16, 2001.

U.S. patent application Ser. No. 09/810,334 filed Mar. 14, 2001.

U.S. patent application Ser. No. 09/799,852 filed Mar. 5, 2001.

U.S. patent application Ser. No. 09/761,337 filed Jan. 16, 2001.

U.S. patent application Ser. No. 09/665,442 filed Sep. 19, 2000.

U.S. patent application Ser. No. 09/658,209 filed Sep. 8, 2000.

U.S. patent application Ser. No. 09/653,664 filed Sep. 1, 2000.

U.S. patent application Ser. No. 09/540,482 filed Mar. 31, 2000.

U.S. patent application Ser. No. 09/237,194 filed Jan. 26, 1999.

U.S. patent application Ser. No. 09/422,046 filed Oct. 10, 1999.

U.S. patent application Ser. No. 60/461,526 filed Apr. 8, 2003.

U.S. patent application Ser. No. 60/461,105 filed Apr. 7, 2003.

U.S. patent application Ser. No. 60/360,708 filed Apr. 4, 2003.

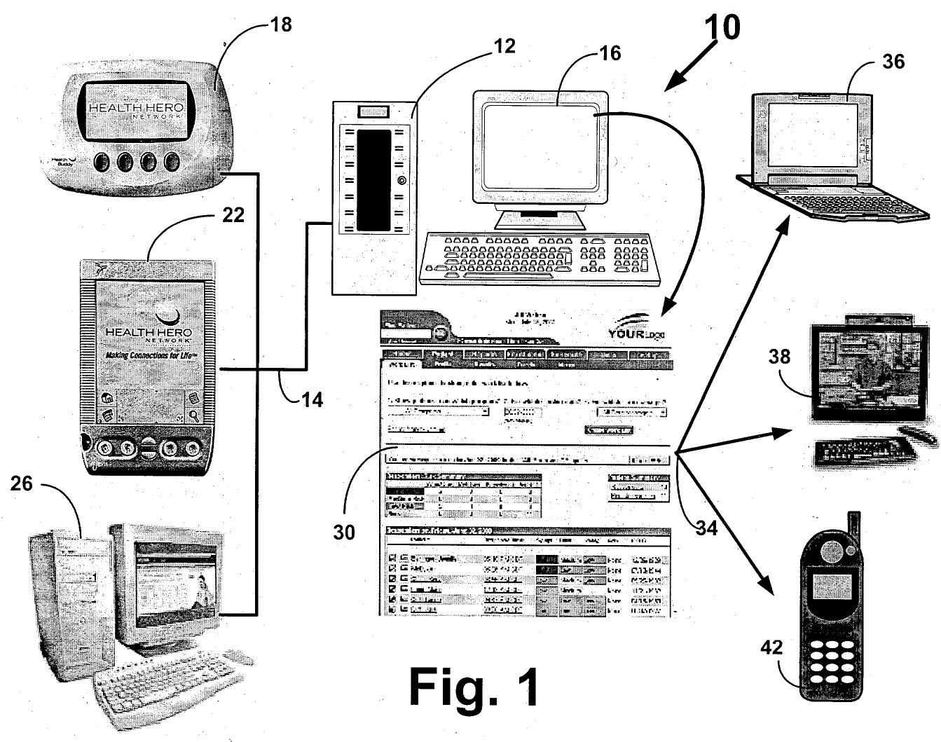

FIG. 1 is a block diagram of a networked system 10 according to a preferred embodiment of the invention. The network system 10 comprises a server 12 that receives input from a remotely programmable device 18 (referred to as a “Health Buddy”), a personal digital assistant (PDA) 22, and a computer 26 via signal communication lines 14. In signal communication with the server 12 is a client station 16 that uses programs to analyze the inputted data, prepare, and output reports 30 via communication lines 34 to output devices represented by a laptop computer 36, a digital television or web TV 38, and a mobile or cellular telephone 42. The digital television is a web TV or microprocessor equivalent peripheral.

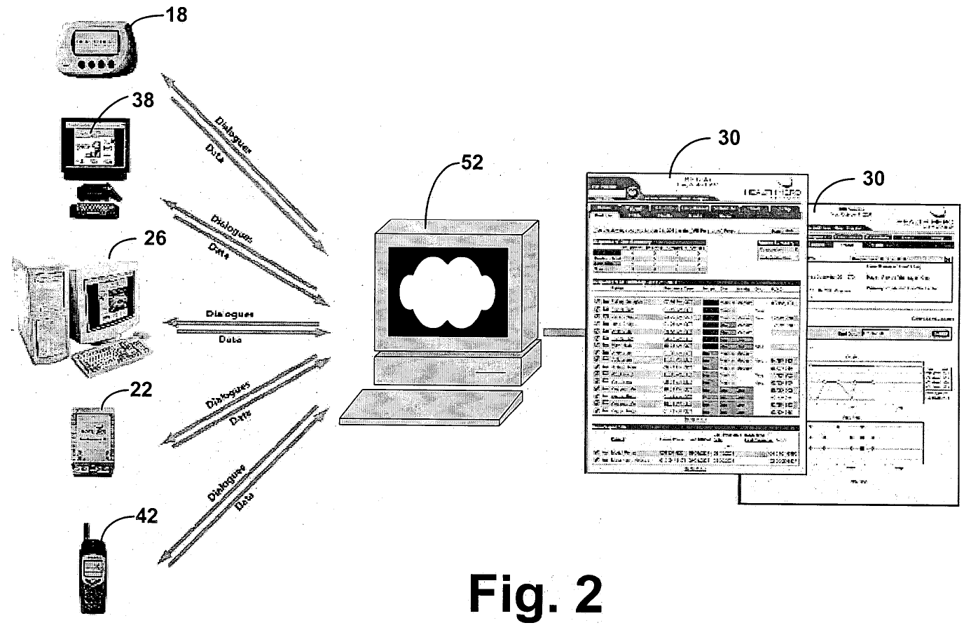

FIG. 2 is a block diagram illustrating the two-way interaction of the components of the system of FIG. 1 using an Internet server 52. Between the Internet server 52, two-way communication occurs between the remotely programmable device 18, the digital television 38, the computer 26, the PDA 22, and the cellular telephone 42. The two-way communication is in the form of data inputted to the Internet server 52 and scripted dialogues sent from the Internet server 52 and the remotely programmable device 18, the digital television 38, the computer 26, the PDA 22, and the cellular telephone 42. The two-way communication also occurs between the laptop computer 36 (not shown). Among the two-way communication sent between the Internet server 52 are the output reports 30.

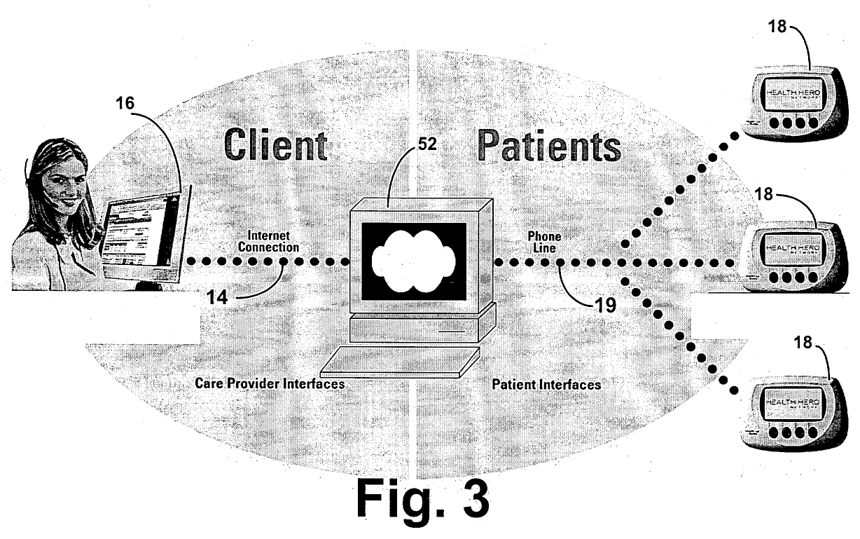

FIG. 3 is a block diagram of a preferred embodiment where a plurality of remotely programmable apparatuses 18 communicate over phone lines 19 (either wired or wireless) to the client station 16 from the Internet server 52 along communication lines 14. Patients located at home or in hospitals and clinics use the remotely programmable apparatus 18 to establish two-way communication with the client station 16.

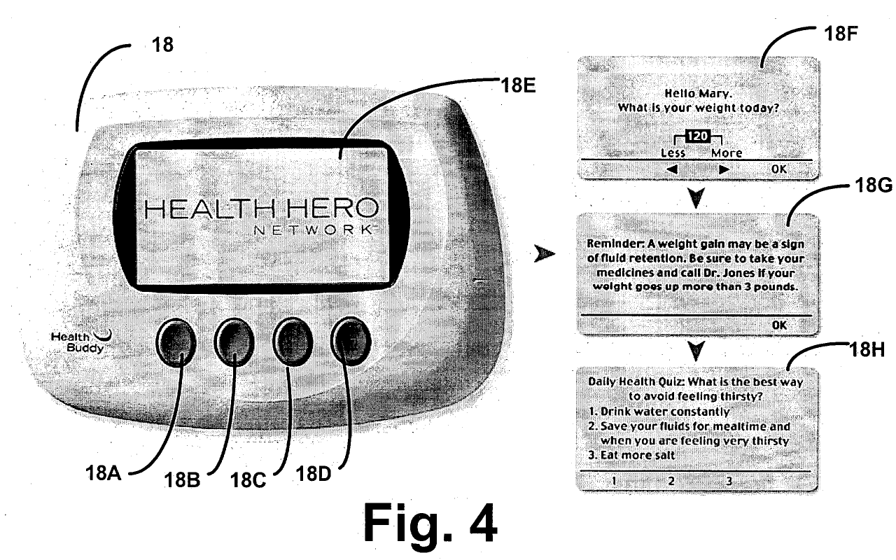

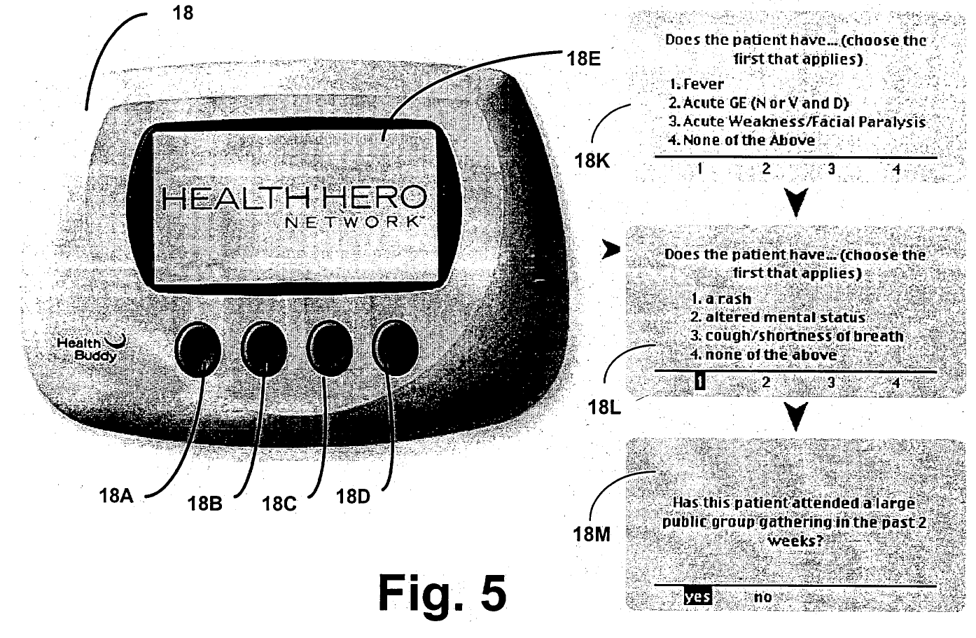

FIG. 4 and FIG. 5 are diagrams that illustrate the remotely programmable device 18 as used in chronic care of a patient in a home setting, and in a hospital setting, respectively, each setting having a series of scripts appropriate for the home, hospital, or clinic setting. The remotely programmable device 18 has a first input button 18A, a second input button 18B, a third input button 18C, a fourth input button 18D, and an alphanumeric display 18E. Each input button 18D either changes the alphanumeric display 18E or issues commands to exchange data and scripts with the client station 16 (not shown). The four user input buttons 18A, 18B, 18C, and 18D are located adjacent to the display 18E and are for entering in the device 18 responses to queries and prompts. In the preferred embodiment, user input buttons 18A, 18B, 18C, and 18D are momentary contact push buttons. In alternative preferred embodiments, the user input buttons 18A, 18B, 18C, and 18D may be replaced by switches, keys, a touch sensitive display screen, or any other data input device.

FIG. 4 shows a representative sampling of home-appropriate scripts as would appear on the display 18E as sent by the client station 16 (not shown) to a home setting. A first script 18F queries “Hello Mary: What is your weight today”. Highlighted in black a patient punches input buttons 18B or C that are aligned with “Less” or “More” as displayed, respectively, until the weight “120”, is reached. Thereafter, “OK” is punched using input button 18D, bringing up a second script 18G that states “Reminder: A weight gain may be a sign of fluid retention. Be sure to take your medicines and call Dr. Jones if your weight goes up more than 2 pounds.” After “OK” is punched, using input button 18D, a third script 18H is presented on the display 18E. The third script provides an educational quiz that states “Daily Health Quiz: What is the best way to avoid feeling thirsty? 1. Drink water constantly, 2. Save your fluids for mealtime and when you are feeling very thirsty; 3. Eat more salt”. Choices 1, 2, and 3, in this illustration, are aligned with the first, second and third input buttons 18 A-C. The patient then uses input button 18A to select answer 1, 18B to select answer 2, or 18C to select an answer 3.

FIG. 5 shows a representative sampling of hospital or clinic-appropriate scripts as would appear on the display 18E as sent by the client station 16 (not shown) to a clinic or hospital situation. A first script 18L queries “Does the patient have . . . (choose the firs that applies) 1. Fever, 2. Acute GE (N or V and D), 3. Acute Weakness/Facial Paralysis”, 4. None of the Above”. The choices 1, 2, 3, and 4 appear at the bottom of first script 18L in a horizontal bar and align respectfully with the first input button 18A, the second input button 18B, the third input button 18C, and the fourth input button 18D. The appropriate input button is pressed depending on the condition of the patient. Thereafter, a second script 18L appears on the display 18E and queries “Does the patient have . . . (choose the first that applies) 1. a rash, 2. altered mental status, 3. cough/shortness of breath, 4. none of the above.” The choices 1, 2, 3, and 4 appear at the bottom of first script 18L in a horizontal bar and align respectfully with the first input button 18A, the second input button 18B, the third input button 18C, and the fourth input button 18D. Highlighted in black choice 1 is made by punching first input button 18A. Thereafter, a third script 18M appears and queries “Has this patient attended a large public group gathering in the past 2 weeks?”, and a “Yes” or “No” are given as the only answer options that are respectively aligned with the first input button 18A and the second input button 18B. The answer “Yes” is selected by pressing the first input button 18A.

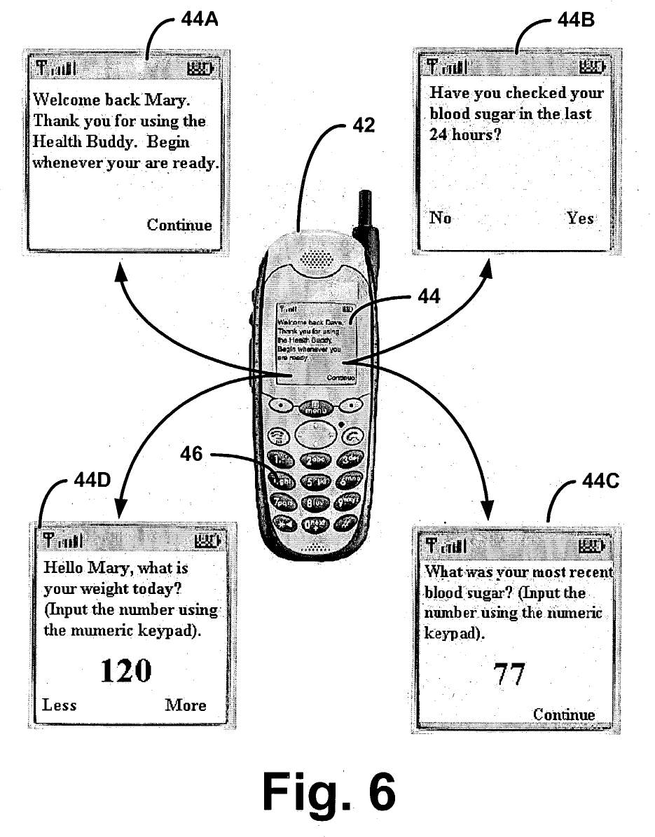

FIG. 6 illustrates a representative set of scripts as they would appear in a preferred embodiment of a cellular or mobile telephone. The scripts are substantially similar to the scripts sent to the remotely programmable device 18. A cellular telephone 42 with an alphanumeric display 44 and a keypad 46 receives a first script 44A from the client station 16 (not shown) that states “Welcome back Mary. Thank you for using the Health Buddy. Begin whenever your are ready. Continue”. An appropriate key is pressed on the keypad 46 for “continue” to be selected. This response brings a second script 44B that queries “Have you checked your blood sugar in the last 24 hours?” with a “No” or “Yes” as the answer options. In this example, the “Yes” answer option is selected via the keypad 18. This brings up a third script 44C that queries “What was your most recent blood sugar? (Input the number using the numeric keypad).” In this example “77” is entered via the keypad 46. At another time, the telephone 44 is sent a fourth query 44D that states “Hello Mary, what is your weight today? (Input the number using the numeric keypad).” In this example, “120” is entered. Optionally, a statement if this weight represents an increase from a previously entered value, an appropriate key assigned to “less” or another key assigned to “more” is pressed depending a given patient's particular situation.

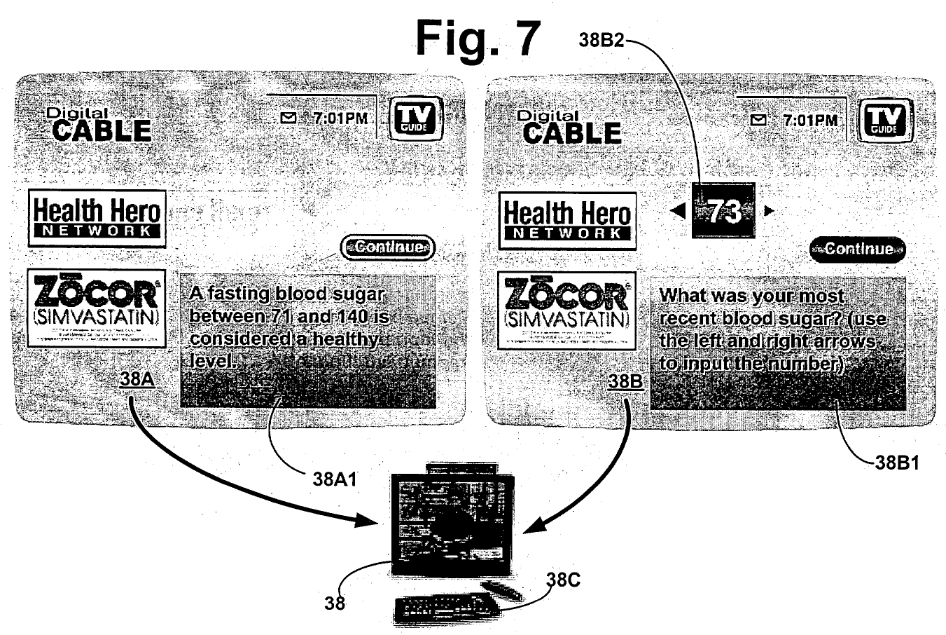

FIG. 7 is an illustration of digital screen shots in a preferred embodiment of the invention using digital television 38. The scripted queries are sent by the client station 16 (not shown) to the digital television 38 with responses provided by the patient via a keyboard 38C. A first screen shot 38A with a first script 38A1 that states “A fasting blood sugar between 71 and 140 is considered a healthy level”. A second screen shot 38B with a second script 38B1 that queries “What was your most recent blood sugar? (Use the left and right arrows to input the number)”, to which, the patient enters “73” at screen location depicted by 38B2 using the keyboard 38C.

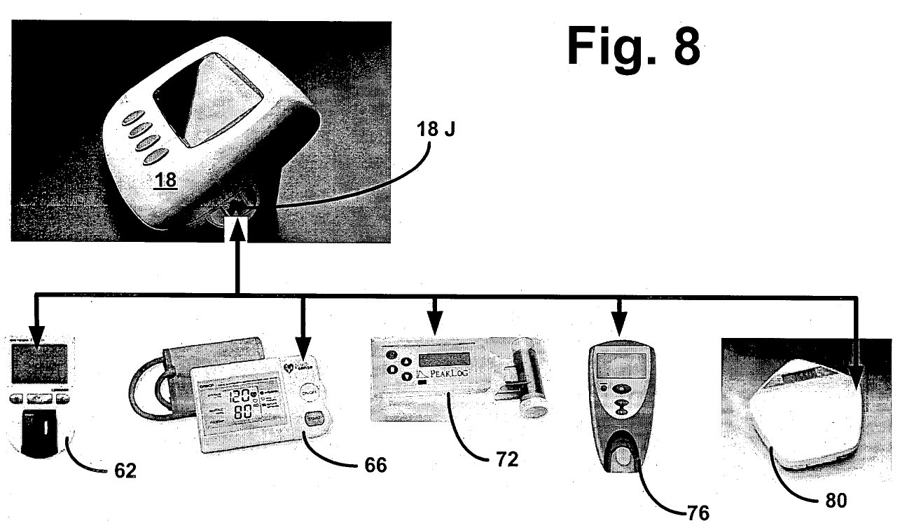

The remotely programmable device 18 is configured to receive multiple medical instruments via standard telephone jacks. FIG. 8 is a representative illustration of the medical devices capable of being attached to the device 18 via a communications port 18J to transmit objectively measured physiologic values. Among the devices include a glucometer 62, a sphygmomanometer 66, a peak flow spirometer 72 to measure lung function, an oxygen sensor 76 to measure blood oxygen levels, and weight balance 80. Other devices (not shown) would be devices that qualitatively or quantitatively report cholesterol, HbA1c, blood coagulation measures, temperature, ECG, cardiac events, ICD devices, other heart pacing devices, EEG, other neurological measures, peak flow, oxygen consumption, other measures of metabolism, sleep measures including waking, breathing, and restlessness (CPAP readings). Other preferred embodiments include multiple medical devices coupled to or is integrated with another medical device which houses interactive functions. For example, a glucometer that couples with an insulin pump enabled with interactive functions, or a spirometry device that couples to a CPAP device enabled with interactive remote monitoring functions. These coupled interactive medical devices send signal transmissions to the communications port 18J.

The remotely programmable device 18 similarly configured to receive input of environmental measurements of which a patient is exposed to, for example, temperature, humidity, presence or absence of chemical agents, biological agents, or radioactivity in the air, water, or food.

Other preferred embodiments of the microprocessor based PDA 22, the computer 26, the laptop computer 36, the cellular telephone 42, and the digital television 38 include receiving scripted queries as described above for the remotely programmable device 18. Furthermore, the PDA 22, the computer 26, the laptop computer 36, the cellular telephone 42, and the digital television 38 are configurable to receive transmissions from the physiologic and environmental measuring devices as similarly communicated to the remotely programmable device 18. Other preferred embodiments of the microprocessor based remotely programmable device 18, the PDA 22, the computer 26, the laptop computer 36, the digital television 38, and the cellular telephone 42 include receiving transmissions from physiologic and environmental measuring devices via Bluetooth or equivalent infra-red or other wireless transmissions.

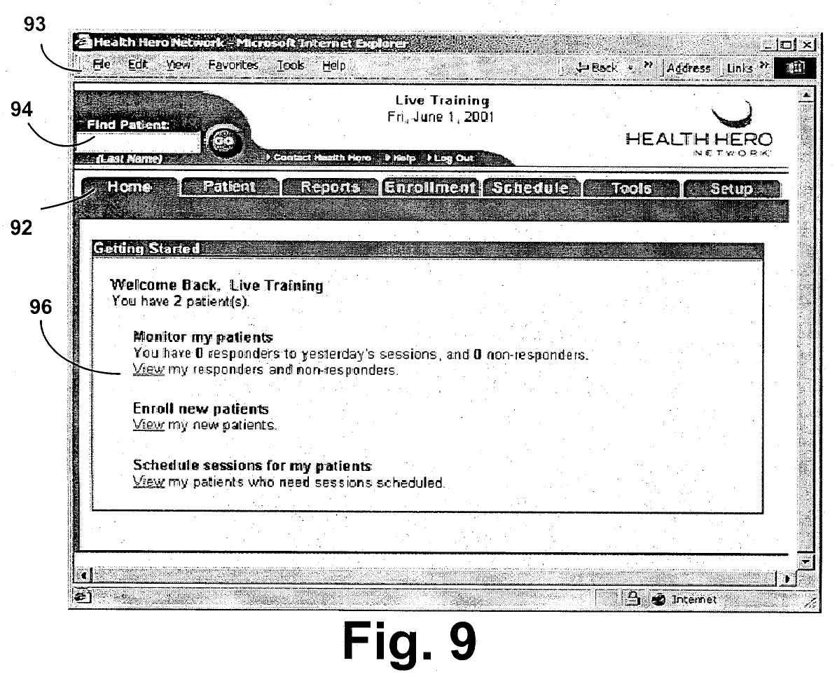

FIGS. 9-21 illustrate web based or Internet or intranet preferred embodiments as presented on monitors or displays of the computer 26, the laptop computer 36, the PDA 22, and the digital television 38. FIG. 9 is a home screen shot 92 as would appear on the client station 16 and that includes a menu bar 93, and a “Live Training-Getting Started” 96 section that gives the manager operating the client station 16 options to either remotely monitor patients, enroll patients, or schedule sessions with patients in communication with the client station 16 via the microprocessor based remotely programmable device 18, the PDA 22, the computer 26, the laptop computer 36, the digital television 38, and the cellular telephone 42. The patient's data is brought up by entering the name of the patient in a “Find Patient” box 94.

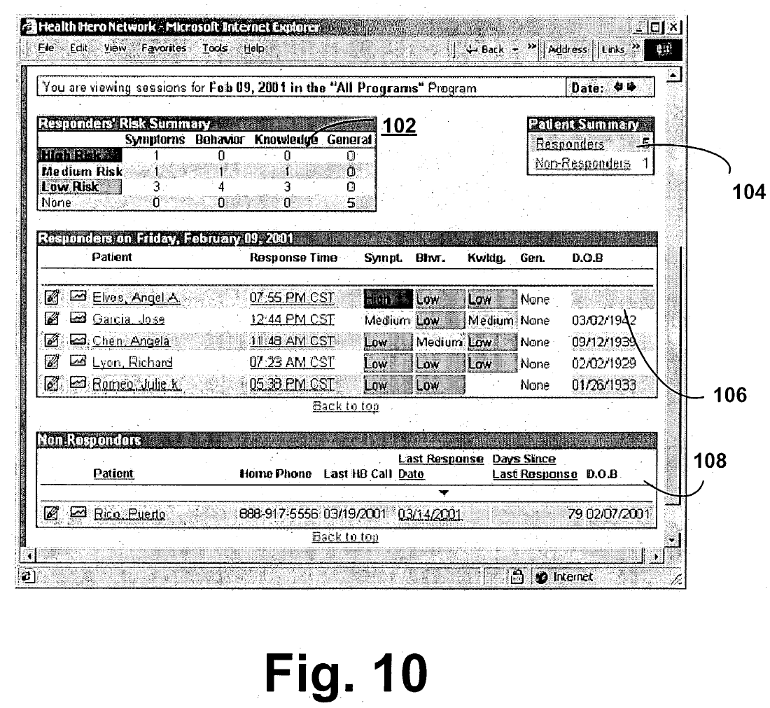

FIG. 10 is screen shot of patient work list as would appear on the client station 16 preferred embodiment of the invention. The patient work list includes a Responders' Risk Summary section 102, a patient summary section 104 that enumerates responders and Non-Responders, a Responders-by-date section 106 with demographic data, and a non-responders-by date section 108 with demographic data. The Responders' Risk Summary is categorized by symptoms, behavior, Knowledge, and General information into High, Medium, Low, and None risk groups.

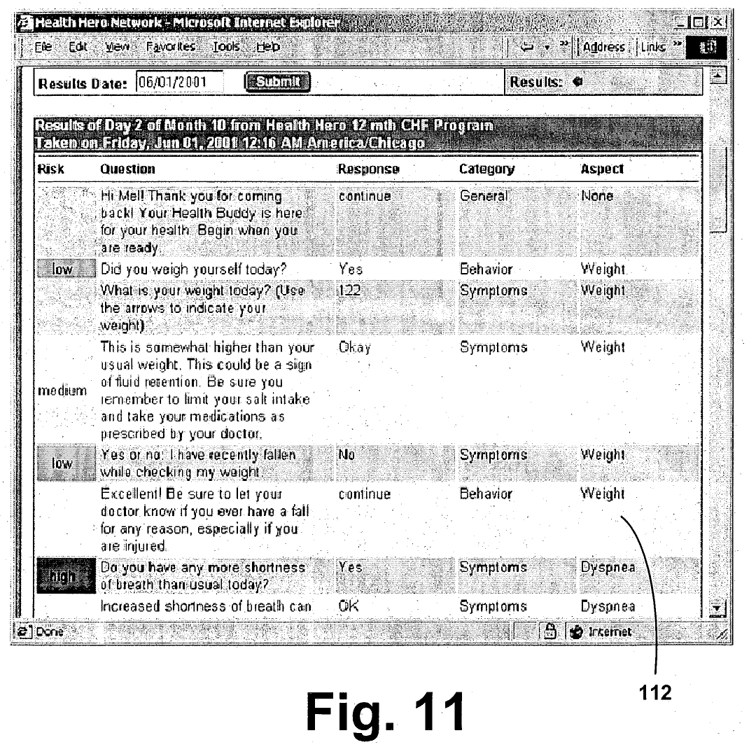

FIG. 11 is a screen shot of a patient results report as would appear at the client station preferred embodiment of the invention. Here answers to queries are itemized in a results table 112 by question, response to the question, the category of question, and the aspect or parameter measured. The answers from a given patient are categorized into none, low, medium, and high risk groups.

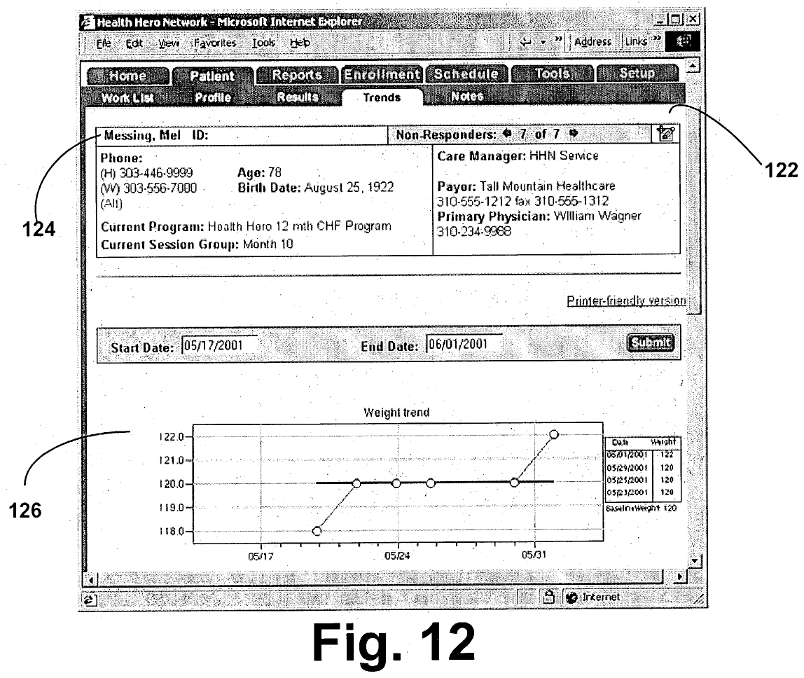

FIG. 12 is a screen shot of a trend report 112 as would appear at the client station 16 preferred embodiment of the invention. Here the trend report 112 includes the name and demographics 124 of a patient and a graphic plot 126 of a measure parameter, in the case, a weight trend by date.

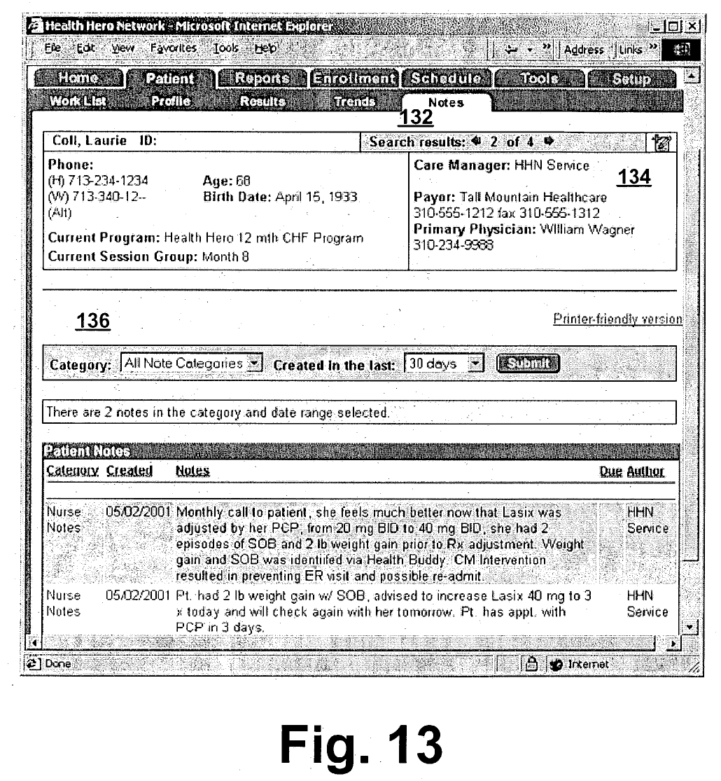

FIG. 13 is a screen shot of patients' notes 132 as would appear on the client station 16 preferred embodiment of the invention. The patients' notes 132 includes a Care Manager section 134 detailing insurance and primary physician data, and a tabular section 136 that lists a compilation of dated notes relevant to the care of patients.

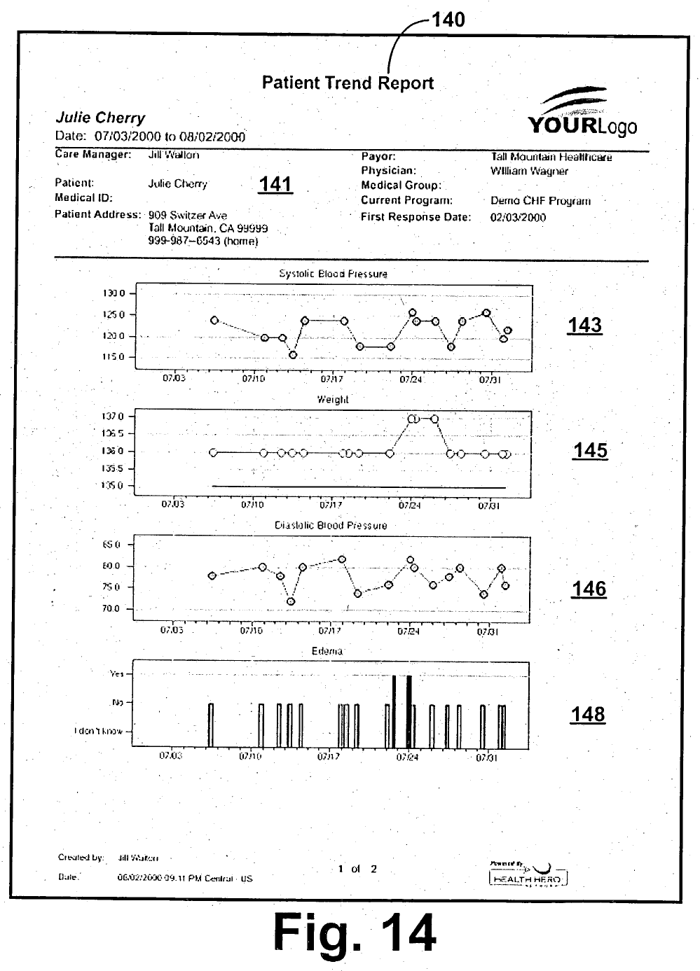

FIG. 14 is a screen shot of a multi parameter trend note 140 of a patient as would appear on the client station 16 preferred embodiment of the invention. The multi parameter trend note 140 includes a patient information section 141 that lists the patient's name, patient's address, physician, medical group, case manager, current program and first response date. The multi parameter trend note includes measurements of several physiologic parameters in graphic format by date. For example, the graph includes a plot of systolic blood pressure 143, a plot of weight 145, a plot of diastolic blood pressure 146, and a plot of edema 148. FIG. 14 is a screen shot of a trend report of an individual patient of the client station preferred embodiment of the invention;

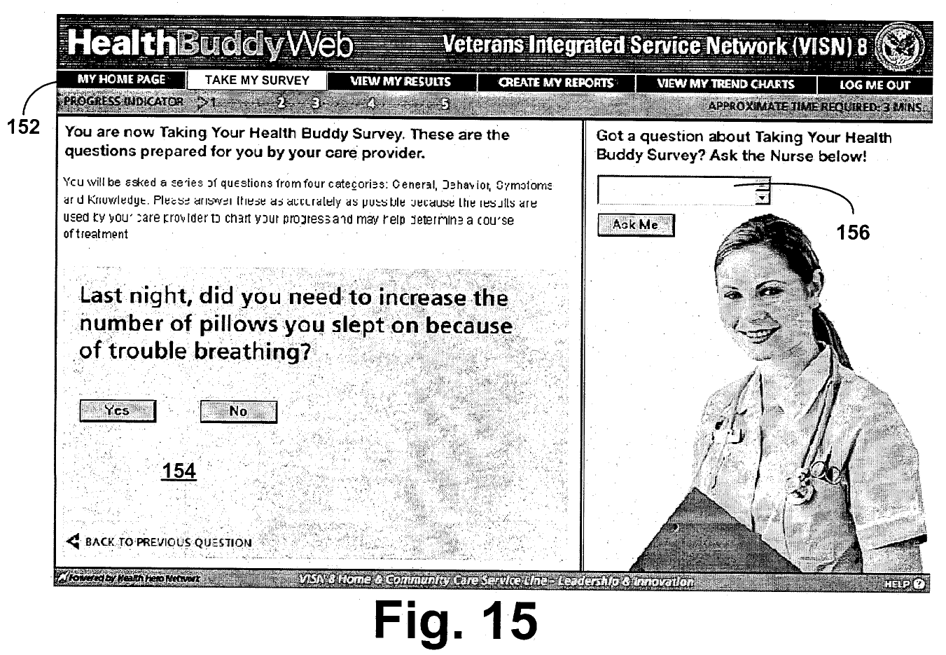

FIG. 15 is a screen shot of a “Take My Survey” 152 section of a home page where a survey questionnaire posed to an individual patient as appearing on the patient's PDA 22, personal computer 26, laptop computer 36, or digital television 38 in a web-preferred embodiment of the invention. A query 154 asking “Last night, did you need to increase number of pillows you slept on because of trouble breathing?—Yes or No” appear on the left side of the screen shot. On the right side of the screen shot is a question entry box 156 with “ask me” entry input button where the patient types in a specific question, or alternatively, selects from an adjacent pull down menu of categorized questions.

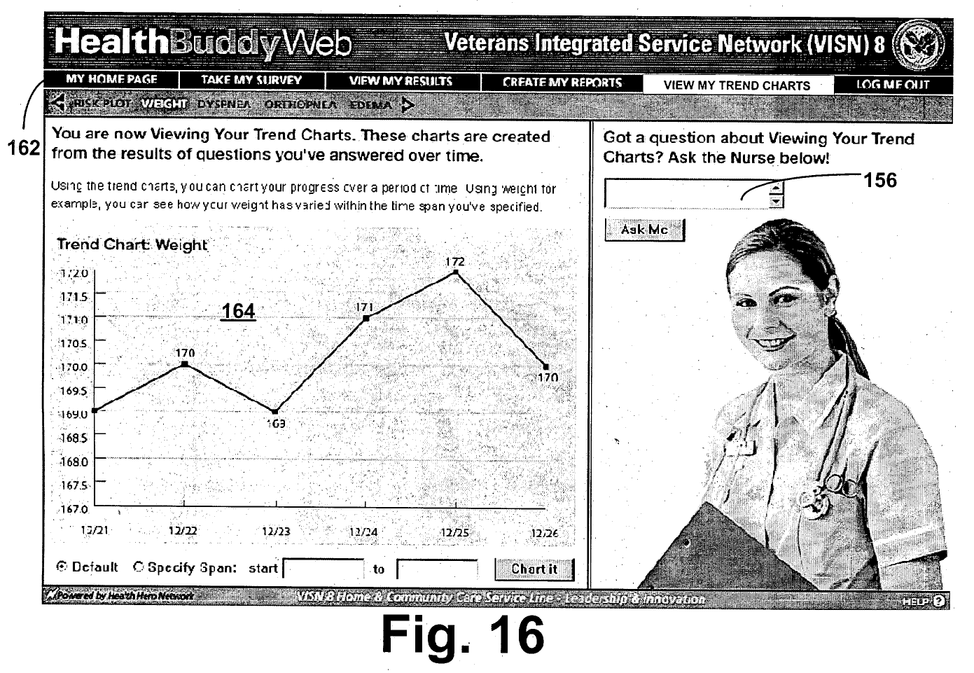

FIG. 16 is a screen shot of a “View My Trend Charts” 162 section of a home page where a trend graphic of a measured parameter of an individual patient is presented as appearing on the patient's PDA 22, personal computer 26, laptop computer .36, or digital television 38 in a web-preferred embodiment of the invention. A trend chart graphic 164 of weight plotted against date is presented on the left side of the screen shot. On the right side of the screen shot is the question entry box 156 with “ask me” entry button where the patient types in a specific question, or alternatively, selects from an adjacent pull down menu of categorized questions.

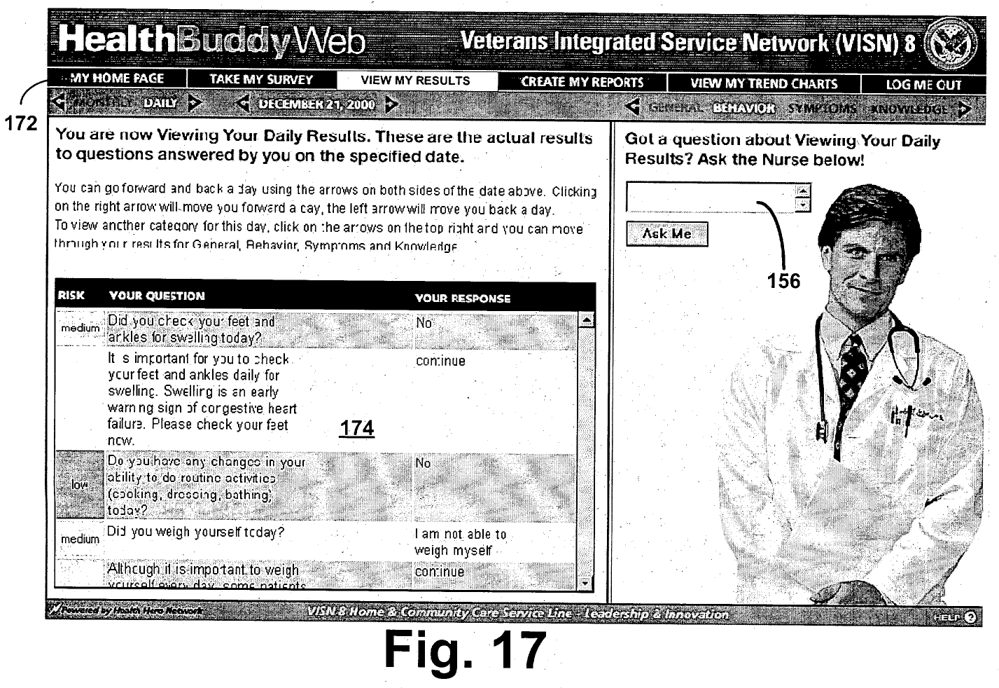

FIG. 17 is a screen shot of a “view my results” report 172 as appearing on the microprocessor-based equipment in the web-preferred embodiment of the invention. On the left side of the screen shot is a table 174 listing the questions sent by the client station 16 (not shown) and responses by the patients, each categorized by risk level. On the right side of the screen shot is the question entry box 156 with “ask me” entry button where the patient types in a specific question, or alternatively, selects from an adjacent pull down menu of categorized questions.

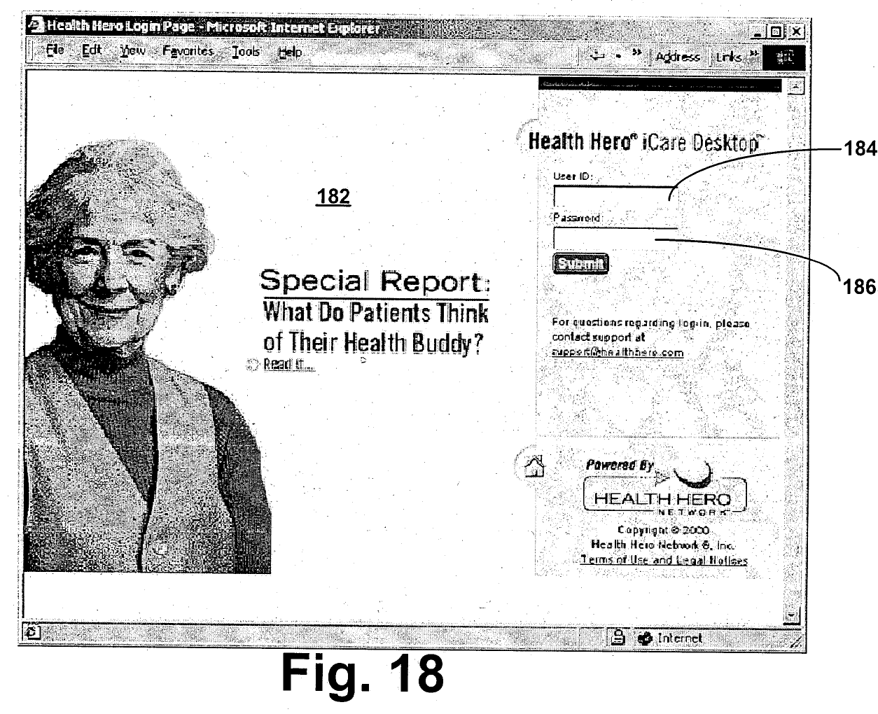

FIG. 18 is a screen shot of a “Special Report” 182 as appearing on the patient's microprocessor-based equipment in the web-preferred embodiment of the invention. A user ID entry box 184 and a Password entry box 186 with submit button is shown on the right side of the screen shot. The password entry box uses a personal identification number (PIN) assigned by the manager of the client station 16 or designate.

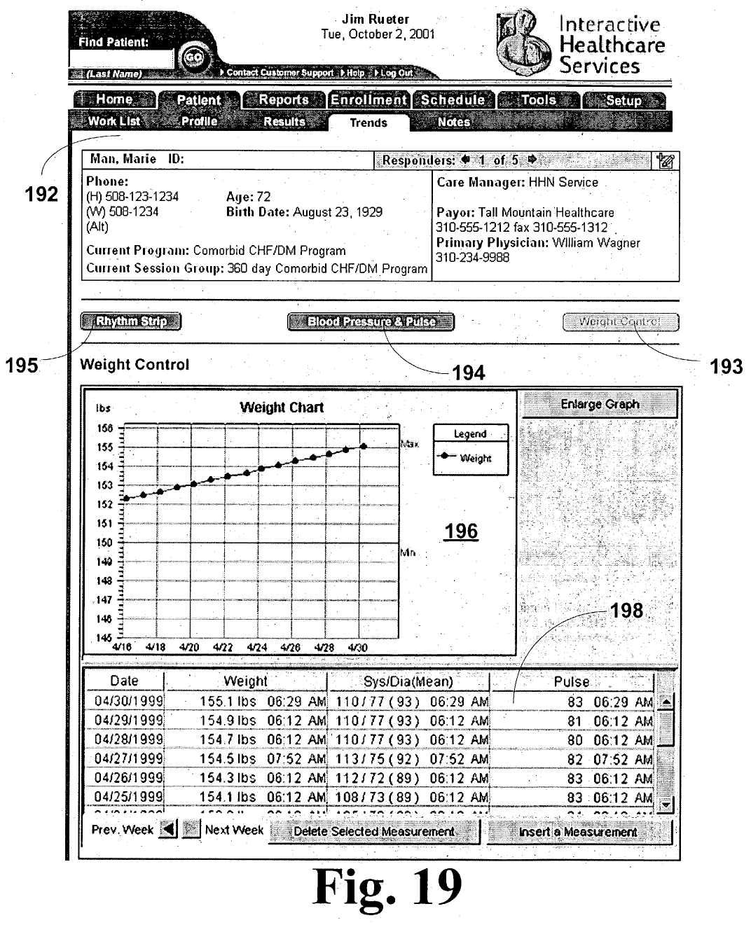

FIG. 19 is a screen shot of a “Trends” report 192 as appearing on the patient's microprocessor-based equipment and the client station in the web-preferred embodiment of the invention. A screen button 193 is pressed to present the trend report 192. Other buttons include a “Blood Pressure and Pulse” button 194, and a “Rhythm Strip” button 195. A weight chart 196 is shown along with a tabular data table 198 having columns of date, weight, Sys/Dia(mean of the systolic and diastolic blood pressure), and Pulse.

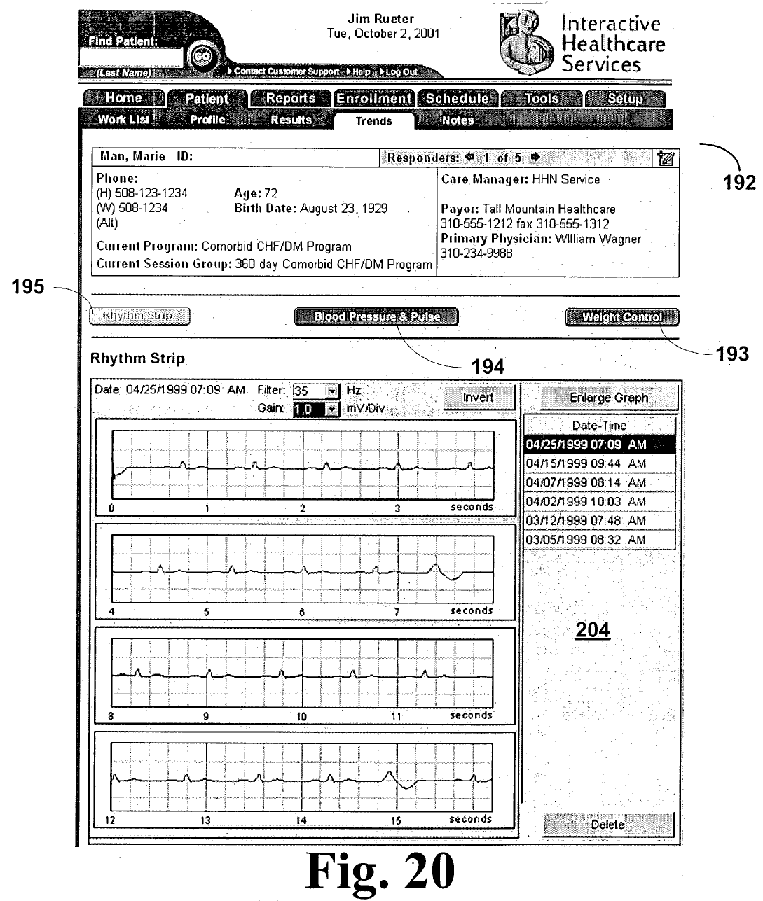

FIG. 20 is a screen shot of a “Trends” report 192 where the “Rhythm Strip” button 195 is pressed to present a panel of EKG readings 204 appearing on the patient's microprocessor-based equipment and the client station 16 in the web-preferred embodiment of the invention.

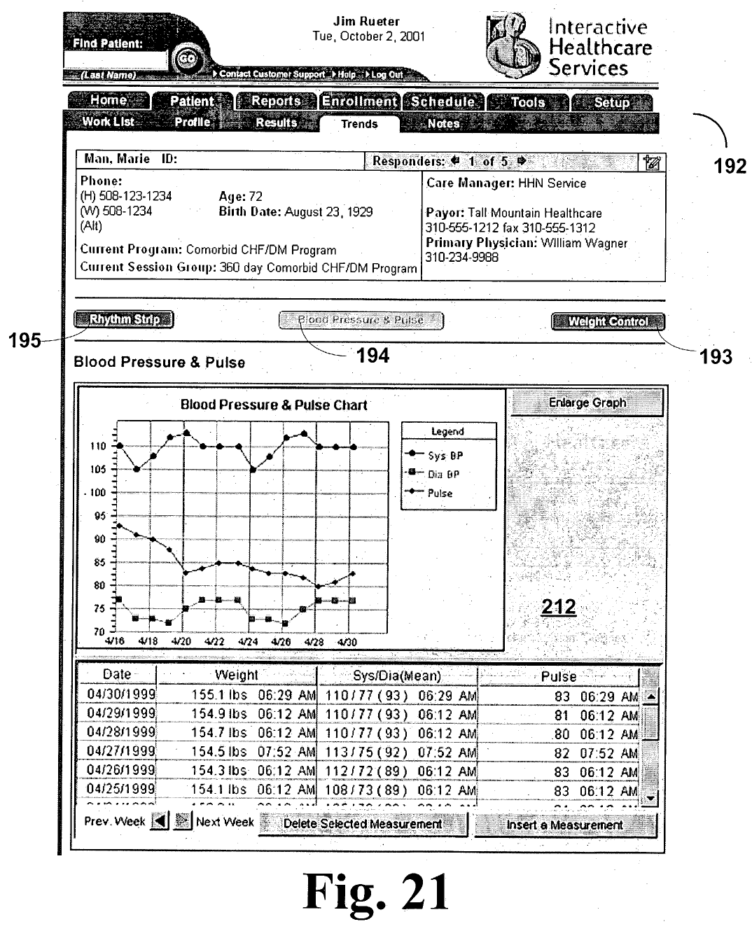

FIG. 21 is a screen shot of a “Trends” 192 as appearing on the patient's microprocessor-based equipment and the client station 16 in the web-preferred embodiment of the invention when the “Blood Pressure and Pulse” button 194 is pressed. A three-line graphic plot 212 is shown in which the systolic blood pressure (top line), the diastolic blood pressure (middle line), and the pulse (bottom line) is shown plotted against date.

While the preferred embodiment of the invention has been illustrated and described, as noted above, many changes can be made without departing from the spirit and scope of the invention. Accordingly, the scope of the invention is not limited by the disclosure of the preferred embodiment.

We claim:

1. A remote monitoring and data management system, the system comprising:
a server having a database;
a client station configured to enter queries into the server;
a plurality of remotely located microprocessor-based apparatuses in communication with the server, each apparatus configured to receive queries from the server, input responses to the queries, and transmit the responses for storage in the database; and
an application microprocessor connected to the client station, the application microprocessor having programs configured to retrieve, process, and analyze, the responses stored in the database for generating reports and intervention protocols.

2. The system of claim 1, wherein the server is a World Wide Web server.

3. The system of claim 1, wherein the client station includes a personal computer.

4. The system of claim 1, wherein each remotely programmable apparatus is connected with a physiologic measuring instrument to measure health-related information of an individual, the health-related information transmitted for storage in the database.

5. The system of claim 4, wherein the health-related information is generated from a glucometer, a sphygmomanometer, a weight balance, an oxygen sensor, and a spirometer.

6. The system of claim 4, wherein the physiologic measuring instrument is connected with a medical device that establishes an interactive function.

7. The system of claim 6, wherein the physiologic measuring instrument is a glucometer and the medical device is an insulin pump.

8. The system of claim 6, wherein the physiologic measuring instrument is a spirometer and the medical device is a CPAP machine.

9. The system of claim 1, wherein, wherein each remotely programmable apparatus is connected with an environmental measuring instrument to measure environmental exposure information associated with an individual, the environmental exposure information transmitted for storage in the database.

10. The system of claim 9, wherein the environmental exposure information is generated from instruments including a thermometer, a chemical detector, a biological agent detector, a radioactivity detector, and a humidity detector.

11. The system of claim 1, wherein the remotely located microprocessor-based apparatuses include a programmable apparatus, a computer, and a cellular telephone.

12. The system of claim 1, wherein each remotely programmable apparatus is a communications device having a user interface for communicating queries and responses, the user interface including a display to present alphanumeric data and user input buttons for entering responses to the displayed alphanumeric data.

13. The system of claim 12, wherein each remotely programmable apparatus further includes a microprocessor configured to execute the queries, the responses to the queries, and to store the identity of the individual responding to the queries.

14. A remote monitoring and data management system, the system comprising:
a server having a database;
a client station configured to enter queries into the server;
a plurality of remotely located microprocessor-based apparatuses in communication with the server, each apparatus configured to receive queries from the server, input responses to the queries, and transmit the responses for storage in the database; and
an application microprocessor connected to the client station, the application microprocessor having programs configured to retrieve, process, and analyze,
the responses stored in the database for generating reports categorizing risk levels and intervention protocols applicable to the risk levels.

15. The system of claim 14, wherein the reports and intervention protocols are accessible by the microprocessor-based apparatuses via World Wide Web.

16. The system of claim 15, wherein the reports further include scripts having a series of queries to diagnose a medical condition, determine the frequency of the medical condition, categorize the medical condition into risk groups, and establish an intervention protocol appropriate for each risk group.

17. The system of claim 15, wherein each remotely programmable apparatus is connected with a physiologic measuring instrument to measure health-related information of an individual, the health-related information transmitted for storage in the database.

18. The system of claim 17, wherein the health-related information is generated from a glucometer, a sphygmomanometer, a weight balance, an oxygen sensor, and a spirometer.

19. The system of claim 17, wherein the physiologic measuring instrument is connected with a medical device that establishes an interactive function.

20. The system of claim 19, wherein the physiologic measuring instrument is a glucometer and the medical device is an insulin pump.

21. The system of claim 19, wherein the physiologic measuring instrument is a spirometer and the medical device is a CPAP machine.

22. The system of claim 19, wherein the reports include graphs of physiologic related measurements.
